(19) United States
(12) Patent Application Publication
(10) Pub. No.: US 2025/0382423 A1
(43) Pub. Date: Dec. 18, 2025

(54) SILICONE OILS, METHODS OF SYNTHESIZING SILICONE OILS FOR INTRAOCULAR LENSES, AND METHODS OF MANUFACTURING FLUID-FILLED INTRAOCULAR LENSES

(71) Applicant: Alcon Inc., Fribourg (CH)

(72) Inventors: William FREEMAN, San Mateo, CA (US); Sean HALENBECK, Palo Alto, CA (US); Keenan BRAYER, San Carlos, CA (US)

(73) Assignee: Alcon Inc., Fribourg (CH)

(21) Appl. No.: 19/227,241

(22) Filed: Jun. 3, 2025

Related U.S. Application Data
(60) Provisional application No. 63/660,977, filed on Jun. 17, 2024.

(51) Int. Cl. C08G 77/38; A61F 2/16

(52) U.S. Cl. CPC C08G 77/38; A61F 2/16

(57) ABSTRACT

Disclosed are ophthalmic silicone oils, methods of synthesizing such silicone oils, and methods of manufacturing fluid-filled intraocular lenses. The ophthalmic silicone oil can comprise branched polymers comprising a cyclosiloxane core and three or more linear polysiloxane arms linked to the cyclosiloxane core. A method of synthesizing the ophthalmic silicone oil can comprise opening a cyclosiloxane ring with a chlorosilane reagent to yield a linear polysiloxane arm and linking the linear polysiloxane arm to a cyclosiloxane core molecule in a hydrosilylation reaction.

### CROSS-REFERENCE TO RELATED APPLICATION

This application claims the benefit of U.S. Patent Application No. 63/660,977 filed on Jun. 17, 2024, the content of which is incorporated herein by reference in its entirety.

### TECHNICAL FIELD

The present disclosure relates generally to the field of fluid-filled intraocular lenses, and, more specifically, to silicone oils, methods of synthesizing silicone oils for intraocular lenses, and methods of manufacturing fluid-filled intraocular lenses.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the crystalline lens matrix from the patient's lens capsule and replaces it with an intraocular lens (IOL).

Newer types of IOLs called accommodating intraocular lenses (AIOLs) may contain fluids within such AIOLs and rely on the movement of such fluids to effect an optical power change in the lens. Silicone oils are examples of fluids that can be used in an AIOL.

An AIOL can be implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flattened (in the anterior-to-posterior direction), which reduces the power of the lens, allowing for distance vision. In this configuration, the patient's native lens is said to be in a disaccommodated state or have undergone disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or have undergone accommodation.

Therefore, any AIOLs implanted within the capsular bag must also possess mechanisms which allow for the base power of the AIOL to increase when the ciliary muscles contract and allow for the base power of the AIOL to decrease when the ciliary muscles relax.

When an AIOL is implanted or otherwise introduced into a patient's native capsular bag, the radially outer portions of the haptics of the AIOL can directly engage with or be in physical contact with the portion of the capsular bag that is connected to the zonules or zonule fibers. Therefore, the haptics can be configured to respond to forces applied radially by the capsular bag when the zonules relax and stretch as a result of ciliary muscle movements.

When the ciliary muscles contract, the peripheral region of the elastic capsular bag reshapes and applies radially inward forces on the radially outer portions of the haptics. The radially outer portions of the haptics then deform or otherwise changes shape and this deformation or shape change causes the volume of the haptic chambers within the haptics to decrease. When the volume of the haptic fluid chambers decreases, the fluid within the haptic fluid chambers is displaced or otherwise pushed into the optic fluid chamber within the optic portion of the AIOL. The optic portion can change shape (increase its curvature) in response to the fluid entering the optic fluid chamber from the haptic fluid chambers. This increases the base power or base spherical power of the AIOL and allows a patient with an implanted AIOL to focus on near objects. In this configuration, the AIOL is said to be in an accommodated state or have undergone accommodation.

When the ciliary muscles relax, the peripheral region of the elastic capsular bag is stretched radially outward and the capsular bag elongates and more room is created within the capsular bag. The radially outer portions of the haptics can be configured to respond to this capsular bag reshaping by returning to its non-deformed or non-stressed configuration. This causes the volume of the haptic fluid chambers to increase or return to its non-deformed volume. This increase in the volume of the haptic fluid chambers causes the fluid within the optic fluid chamber to be drawn out of the optic fluid chamber and back into the haptic fluid chambers. The optic portion can change shape (decrease its curvature or become flatter) in response to the fluid exiting the optic fluid chamber and into the haptic fluid chambers. This decreases the base power or base spherical power of the AIOL and allows a patient with an implanted AIOL to focus on distant objects or provide for distance vision. In this configuration, the AIOL is said to be in a disaccommodated state or have undergone disaccommodation.

The ability to rapidly focus (e.g., switch from distance vision to near vision, or vice versa) is important, for instance, when a subject glances between the road and a vehicle's dashboard while driving. For fluid-driven IOLs, such as AIOLs, the rate of focus or accommodation is inversely proportional to the viscosity of the fluid within such lenses. Therefore, one way of improving the accommodative response of an AIOL is to decrease the viscosity of the fluid (e.g., silicone oil) within the AIOL. For silicone oils in particular, this often means decreasing the average molecular weight of the polymers making up such oils. However, doing so can adversely affect the optical quality of the AIOL as the lower molecular weight polymers of the silicone oil can swell the bulk polymeric material used to make the optic portion and haptics of the AIOL.

The optic portion and haptics of the AIOL are often made using a bulk polymerization process such as a bulk acrylate polymerization process. In general, bulk polymerization converts small monomers into polymers without solvent. While bulk polymerization produces mostly high molecular weight polymers, it also produces a small amount of lower molecular weight oligomers and unreacted monomers. These oligomers are not bonded to the bulk polymer and are simply trapped inside the bulk polymer. If these non-bonded materials were allowed to remain in the lens, they would gradually migrate out and appear as an oily coating on the lens surface known as “polymer bloom.”

While these oligomers and unreacted monomers can be removed through various processes, their removal can leave voids or holes in the polyacrylate where the lower molecular weight molecules used to reside. When the lens is filled with a fluid such as silicone oil, the holes allow small oil molecules to enter and swell the polyacrylate lens material. For example, these small silicone oil molecules can have a molecular weight of less than 2000 Daltons (as measured against polystyrene standards using gel-permeation chromatography). In some instances, the fluid-filled lenses can swell enough to shift lens power by several diopters.

While purifying the silicone oil using a purification process such as solvent fractionation can address the lens swelling problem, such purification processes can often create other unintended consequences that adversely affect the lens. For example, purifying the silicone oil using solvent fractionation can increase the average molecular weight of the silicone oil as lower molecular weight components of the oil are removed through fractionation. Since the viscosity of linear chain silicone oils is directly proportional to the average molecular weight of such oils, the viscosity of such oils can increase once the lower molecular weight components are removed. This inevitably decreases the rate of focus or accommodation of lenses filled with such oils.

Therefore, an improved silicone oil is needed which address the aforementioned problems. Such an oil should have an average molecular weight large enough so that the oil does not swell the bulk polymeric lens material and adversely affect the lens power or focal length. Moreover, the average molecular weight of the oil should be low enough to allow the silicone oil to move and flow so that the lens can perform its accommodative functions. 21 Furthermore, the silicone oil should be produced using a method that is not overly complicated or cost-prohibitive.

### SUMMARY

Disclosed herein are silicone oils and methods of manufacturing silicone oils for intraocular lenses (IOLs). In some embodiments, the silicone oils can be used in an accommodating intraocular lens (AIOL) that relies on fluid movement to effect optical power changes in the AIOL. The silicone oils disclosed herein can also be used in a non-accommodating fluid-adjustable intraocular lens.

In one aspect, an ophthalmic silicone oil is disclosed comprising polymers with a branched polymer structure (or branched polymers). At least one of the branched polymers can comprise a cyclosiloxane core and three or more linear polysiloxane arms linked to the cyclosiloxane core.

In one embodiment, the cyclosiloxane core can comprise a cyclotrisiloxane ring. In another embodiment, the cyclosiloxane core can comprise a cyclotetrasiloxane ring.

In some embodiments, at least one of the linear polysiloxane arms can comprise a linear trisiloxane backbone. In other embodiments, at least one of the linear polysiloxane arms can comprise a linear tetrasiloxane backbone.

In some embodiments, the branched polymers can be substantially X-shaped or shaped as pseudo-dendrimers.

The ophthalmic silicone oil can have a viscosity of less than about 2400 centipoise (cps). For example, the ophthalmic silicone oil can have a viscosity of between about 1000 cps to about 2400 cps, as measured at 25° C.

In some embodiments, the ophthalmic silicone oil can have a refractive index of less than 1.53. In these and other embodiments, the refractive index of the ophthalmic silicone oil can be index matched with the refractive index of a bulk polymeric material making up the IOL.

Also disclosed is a branched polymer comprising a core ring structure comprising a plurality of ring segments and a polysiloxane arm (Rarm) linked to each of the ring segments. Each of the ring segments can have a structure corresponding to Formula I below:

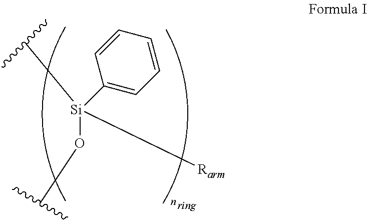

wherein n_{ring} is an integer ≥3 and wherein Rₐᵣₘ has a structure corresponding to Formula II below:

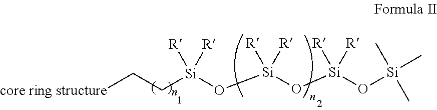

wherein n₁ is an integer=1 or 2, n₂ is an integer=1 or 2, and wherein R′ is a phenyl group or a methyl group.

In some embodiments, the ratio of methyl groups to phenyl groups can be about 2:1.

Also disclosed is a method for synthesizing an ophthalmic silicone oil comprising branched polymers. The method can comprise opening a cyclosiloxane ring with a chlorosilane reagent to yield a linear polysiloxane arm. In some embodiments, the cyclosiloxane ring can be a diphenyl tetramethyl cyclotrisiloxane.

The method can further comprise linking the linear polysiloxane arm to a cyclosiloxane core molecule in a hydrosilylation reaction. In some embodiments, the cyclosiloxane core molecule can be a cyclotetrasiloxane. In other embodiments, the cyclosiloxane core molecule can be a cyclotrisiloxane.

In certain embodiments, the chlorosilane reagent can be an allylchlorodimethylsilane and the ring opening reaction is performed at a reaction temperature of between about 40° C. and 50° C. In these embodiments, the step of opening the cyclosiloxane ring can yield an intermediate linear polysiloxane. The method can further comprise reacting the intermediate linear polysiloxane with a Grignard reagent or a silanolate reagent to yield the linear polysiloxane arm. In certain embodiments, the silanolate reagent can be a lithium trimethylsilanolate.

In other embodiments, the chlorosilane reagent can be a chlorotrimethylsilane and the ring opening reaction can be performed at a reaction temperature of between about 40° C. and 50° C. In these embodiments, the step of opening the cyclosiloxane ring can also yield an intermediate linear polysiloxane. The method can further comprise reacting the intermediate linear polysiloxane with a silanolate reagent to yield the linear polysiloxane arm. In certain embodiments, the silanolate reagent can be a potassium dimethyl (vinyl) silanolate.

Also disclosed is a method of manufacturing a fluid-filled intraocular lens. The method can comprise submerging an unfilled intraocular lens in acetone. The intraocular lens can be made in part of a polyacrylate material. The intraocular lens can comprise an optic fluid chamber and at least one peripheral fluid chamber. The method can further comprise removing the intraocular lens from the acetone and decanting the acetone from the intraocular lens.

The method can also comprise introducing a silicone oil made of branched polymers into at least one of the optic fluid chamber and the at least one peripheral fluid chamber. In some embodiments, the branched polymers of the silicone oil can be substantially X-shaped. The silicone oil can have a viscosity between about 1000 cps to 2400 cps, as measured at 25° C. The silicone oil can have a refractive index of less than 1.53.

In some embodiments, the step of introducing the silicone oil can comprise injecting the silicone oil into at least one of the optic fluid chamber and the at least one peripheral fluid chamber.

In certain embodiments, at least one of the branched polymers of the silicone oil can comprise a cyclosiloxane core and three or more linear polysiloxane arms linked to the cyclosiloxane core.

In some embodiments, the cyclosiloxane core can comprise a cyclotrisiloxane ring. In other embodiments, the cyclosiloxane core can comprise a cyclotetrasiloxane ring.

In some embodiments, at least one of the linear polysiloxane arms can comprise a linear trisiloxane backbone. In other embodiments, at least one of the linear polysiloxane arms can comprise a linear tetrasiloxane backbone.

### BRIEF DESCRIPTION OF THE DRAWINGS

FIG. 1A illustrates a top plan view of one embodiment of an accommodating intraocular lens (AIOL). The AIOL can rely on the internal displacement of the silicone oil disclosed herein to effect an optical power change in the AIOL.

FIGS. 1B and 1C illustrate sectional views of the AIOL of FIG. 1A.

FIG. 2 illustrates one embodiment of a method of synthesizing an ophthalmic silicone oil comprising branched polymers.

FIG. 3A illustrates example reaction steps for preparing arms of the branched polymer.

FIG. 3B illustrates additional example reaction steps for preparing arms of the branched polymer.

FIG. 3C illustrates examples of other cyclosiloxanes that can be used as monomers or substrates for the ring-opening polymerization reactions.

FIG. 4 illustrates another example of reaction steps for preparing arms of the branched polymer.

FIG. 5 illustrates different embodiments of cyclosiloxane core molecules.

FIG. 6 illustrates examples of hydrosilylation reactions linking the linear polysiloxane arms to the cyclosiloxane core.

FIG. 7A illustrates one embodiment of a substantially X-shaped branched polymer synthesized using the methods disclosed herein.

FIG. 7B illustrates another embodiment of a substantially X-shaped branched polymer synthesized using the methods disclosed herein.

FIG. 8 illustrates one embodiment of a method of manufacturing a fluid-filled IOL.

### DETAILED DESCRIPTION

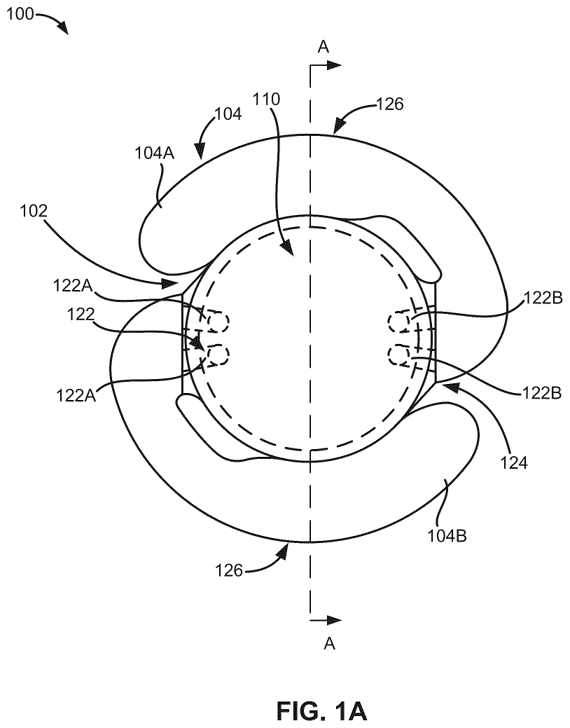

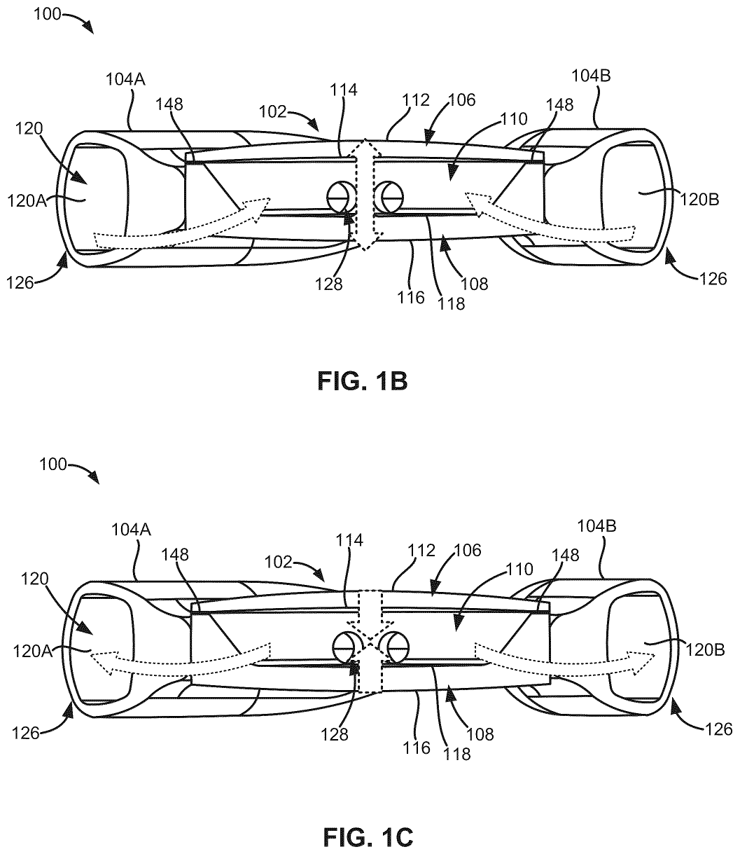

Disclosed herein are silicone oils and methods of manufacturing silicone oils for use in intraocular lenses. In some embodiments, the silicone oils can be used in an accommodating intraocular lens (AIOL) that relies on internal fluid displacement to effect optical power changes in the AIOL. For example, the silicone oils disclosed herein can be used with the AIOL shown in FIGS. 1A-1C. Moreover, the silicone oils disclosed herein can be used with the AIOLs disclosed in the following U.S. patent applications and publications: U.S. patent application Ser. No. 17/060,901 filed on Oct. 1, 2020 and U.S. patent application Ser. No. 17/060,919 filed on Oct. 1, 2020; U.S. Pat. Pub. No. 2020/0337833; U.S. Pat. Pub. No. 2018/0256315; U.S. Pat. Pub. No. 2018/0153682; and U.S. Pat. Pub. No. 2017/0049561 and in the following issued U.S. patents: U.S. Pat. Nos. 10,299,913; 10,195,020; and 8,968,396, the contents of which are incorporated herein by reference in their entireties.

The silicone oils disclosed herein can also be used in a non-accommodating fluid-adjustable intraocular lens. For example, the silicone oils disclosed herein can also be used in the non-accommodating fluid-adjustable intraocular lens disclosed in U.S. patent application Ser. No. 17/060,940 filed on Oct. 1, 2020.

FIG. 1A illustrates a top plan view of an embodiment of an AIOL 100 that can be

implanted within a subject and that rely on the internal displacement of silicone oils disclosed herein to effect an optical power change in the AIOL 100. The AIOL 100 can comprise an optic portion 102 and one or more haptics 104 including a first haptic 104A and a second haptic 104B coupled to and extending peripherally from the optic portion 102.

When implanted within the native capsular bag, the optic portion 102 can be adapted to refract light that enters the eye onto the retina. The one or more haptics 104 can be configured to engage the capsular bag and be adapted to deform in response to ciliary muscle movement (e.g., muscle relaxation, muscle contraction, or a combination thereof) in connection with capsular bag reshaping.

FIGS. 1B and 1C illustrate sectional views of the AIOL 100 taken along cross-section A-A of FIG. 1A. As shown in FIGS. 1B and 1C, the optic portion 102 can comprise an anterior element 106 and a posterior element 108. A fluid-filled optic fluid chamber 110 can be defined in between the anterior element 106 and the posterior element 108.

The anterior element 106 can comprise an anterior optical surface 112 and an anterior inner surface 114 opposite the anterior optical surface 112. The posterior element 108 can comprise a posterior optical surface 116 and a posterior inner surface 118 opposite the posterior optical surface 116. Any of the anterior optical surface 112, the posterior optical surface 116, or a combination thereof can be considered and referred to as an external optical surface. The anterior inner surface 114 and the posterior inner surface 118 can face the optic fluid chamber 110. At least part of the anterior inner surface 114 and at least part of the posterior inner surface 118 can serve as chamber walls of the optic fluid chamber 110.

Each of the one or more haptics 104 can comprise a haptic fluid chamber 120 within the haptic 104. For example, the first haptic 104A can comprise a first haptic fluid chamber 120A within the first haptic 104A and the second haptic 104B can comprise a second haptic fluid chamber 120B within the second haptic 104B. The haptic fluid chamber 120 (e.g., any of the first haptic fluid chamber 120A, the second haptic fluid chamber 120B, or a combination thereof) can be in fluid communication with or fluidly connected to the optic fluid chamber 110.

The optic fluid chamber 110 can be in fluid communication with the one or more haptic fluid chambers 120 through a pair of fluid channels 122 (see FIG. 1A). The fluid channels 122 can be conduits or passageways fluidly connecting the optic fluid chamber 110 to the haptic fluid chamber 120. The pair of fluid channels 122 can be spaced apart from one another. 3

In some embodiments, the pair of fluid channels 122 can be defined and extend through part of the optic portion 102. More specifically, the pair of fluid channels 122 can be defined and extend through the posterior element 108.

FIG. 1A illustrates that one or more haptics 104 can be coupled to the optic portion 102 at a haptic-optic interface 124. For example, the one or more haptics 104 can be coupled to the 8 optic portion at a reinforced portion along the optic portion 102. The reinforced portion can be part of the haptic-optic interface 124. The pair of fluid channels 122 can be defined or formed within part of the reinforced portion.

The optic fluid chamber 110 can be in fluid communication with the first haptic fluid chamber 120A through a first pair of fluid channels 122A. The optic fluid chamber 110 can also be in fluid communication with the second haptic fluid chamber 120B through a second pair of fluid channels 122B.

In some embodiments, the first pair of fluid channels 122A and the second pair of fluid channels 122B can be positioned substantially on opposite sides of the optic portion 102. The first pair of fluid channels 122A can be positioned substantially diametrically opposed to the second pair of fluid channels 122B. 19

The first pair of fluid channels 122A and the second pair of fluid channels 122B can be defined or extend through part of the optic portion 102. The first pair of fluid channels 122A and the second pair of fluid channels 122B can be defined or extend through the posterior element 108.

The pair of fluid channels 122 (e.g., any of the first pair of fluid channels 122A or the second pair of fluid channels 122B) can have a pair of inner apertures 128 disposed at one end of the fluid channels 122 and another pair of outer apertures disposed at the other end of the fluid channels 122. The pair of inner apertures 128 can be defined or formed on part of the posterior element 108.

Fluid (e.g., silicone oil) within the haptic fluid chamber 120 can flow out of the haptic fluid chamber 120 and into the optic fluid chamber 110 via the pair of fluid channels 122 when the haptic 104 is coupled to the optic portion 102. Similarly, fluid within the optic fluid chamber 110 can flow out of the optic fluid chamber 110 through the pair of fluid channels 122 and into the haptic fluid chamber 120.

Each of the haptics 104 can also comprise a radially outer portion 126 configured to face and contact an inner surface of a patient's capsular bag when the AIOL 100 is implanted within the capsular bag. Engagement of the capsular bag with the radially outer portion 126 of the haptics 104 will be discussed in more detail in the following sections.

The optic portion 102 can have a base power or base spherical power. The base power of the optic portion 102 can be configured to change based on an internal fluid pressure within the fluid-filled optic fluid chamber 110. The base power of the optic portion 102 can be configured to increase or decrease as fluid enters or exits the fluid-filled optic fluid chamber 110.

The base power of the optic portion 102 can be configured to increase as fluid enters the fluid-filled optic fluid chamber 110 from the haptic fluid chamber(s) 120, as shown in FIG. 1B. The base power of the optic portion 102 can be configured to decrease as fluid exits or is drawn out of the fluid-filled optic fluid chamber 110 into the haptic fluid chamber(s) 120, as shown in FIG. 1C.

It should be noted that although FIG. 1B illustrates the fluid entering the optic fluid chamber 110 from the haptic fluid chambers 120 using the curved broken-line arrows, fluid enters the optic fluid chamber 110 via the fluid channels 122 (including through the inner apertures 128) and haptic fluid ports. It should also be noted that although FIG. 1C illustrates the fluid exiting the optic fluid chamber 110 into the haptic fluid chambers 120 using the curved broken-line arrows, fluid exits the optic fluid chamber 110 via the fluid channels 122 (including through the inner apertures 128) and haptic fluid ports.

In some embodiments, the optic portion 102 can be made in part of a deformable or flexible polymeric material. For example, the anterior element 106, the posterior element 108, or a combination thereof can be made in part of a deformable or flexible polymeric material.

The one or more haptics 104 (e.g., the first haptic 104A, the second haptic 104B, or a combination thereof) can be made in part of the same deformable or flexible material as the optic portion 102. In other embodiments, the one or more haptics 104 can be made in part of different materials from the optic portion 102.

In some embodiments, the optic portion 102 can comprise or be made in part of a bulk polymeric material. The bulk polymeric material can be a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof. The term “acrylate” as used herein can be understood to refer to acrylates, methacrylates, or a combination thereof interchangeably unless otherwise specified.

The cross-linked copolymer used to make the lens body material can also comprise a cross-linker or cross-linking agent such as ethylene glycol dimethacrylate (EGDMA). For example, the cross-linked copolymer used to make the lens body material can also comprise a cross-linker or cross-linking agent (e.g., EGDMA). The cross-linked copolymer used to make the lens body material can also comprise an initiator or initiating agent (e.g., Perkadox 16) and a UV absorber.

The haptic(s) 104 can comprise or be made in part of the same bulk polymeric material or another type of polymeric material. The haptic(s) 104 can be made in part of a cross-linked copolymer comprising a copolymer blend. In some embodiments, the copolymer blend can comprise an alkyl acrylate, a fluoro-alkyl acrylate, and a phenyl-alkyl acrylate. The cross-linked copolymer used to make the haptic material can also comprise a cross-linker or cross-linking agent, such as EGDMA. The cross-linked copolymer used to make the haptic material can also comprise a number of photoinitiators or photoinitiating agents (e.g., camphorquinone, 1-phenyl-1,2-propanedione, and 2-ethylhexyl-4-(dimenthylamino) benzoate).

In some embodiments, the refractive index of the bulk polymeric material can be between about 1.48 and about 1.53. In certain embodiments, the refractive index of the bulk polymeric material can be between about 1.50 and about 1.53.

The optic portion 102 can be configured to deform, flex, or otherwise change shape in response to fluid entering or exiting the optic fluid chamber 110. The haptic(s) 104 can also be configured to deform or otherwise change shape in response to interactions or engagement with the capsular bag of a patient when the AIOL 100 is implanted within an eye of the patient.

The base power of the optic portion 102 can be configured to change in response to the shape change undertaken by the shape-changing components of the optic portion 102 (e.g., the anterior element 106, the posterior element 108, or a combination thereof). The optic portion 102 can be configured to change shape in response to a physiologic muscle movement (e.g., ciliary muscle movement) undertaken by a patient when the AIOL 100 is implanted within a capsular bag of the eye of the patient. The AIOL 100 can deform or change shape in response to ciliary muscle movements related capsular bag reshaping.

For example, when the AIOL 100 is implanted into a patient's native capsular bag, the radially outer portions 126 of the haptics 104 of the AIOL 100 can directly engage with or be in physical contact with the portion of the capsular bag that is connected to the zonules or zonule fibers. Therefore, the radially outer portions 126 of the haptics 104 can be configured to respond to capsular bag reshaping forces that are applied radially when the patient's zonules relax and stretch as a result of ciliary muscle movements.

When the ciliary muscles contract, the peripheral region of the elastic capsular bag reshapes and applies radially inward forces on the radially outer portions 126 of the haptics 104 (for example, the elastic capsular bag applies radially inward forces on the radially outer portion 126 of the first haptic 104A and on the radially outer portion 126 of the second haptic 104B). The radially outer portions 126 of the haptics 104 can then deform or otherwise change shape and this deformation or shape change causes the volume of the haptic fluid chambers to decrease. When the volume of the haptic fluid chambers 120 decreases, the fluid within the haptic fluid chambers 120 is moved or pushed into the optic fluid chamber 110 within the optic portion 102.

The optic portion 102 can change shape (increase its curvature) in response to the fluid entering the optic fluid chamber 110 from the haptic fluid chambers 120. This increases the base power or base spherical power of the AIOL 100 and allows a patient with the AIOL 100 implanted within the eye of the patient to focus on near objects. The AIOL 100 can also be considered to be in an accommodated state or have undergone accommodation. 27

When the patient's ciliary muscles relax, the peripheral region of the elastic capsular bag is stretched radially outward and the capsular bag elongates and more room is created within the capsular bag. The radially outer portions 126 of the haptics 104 can be configured to respond to this capsular bag reshaping by returning to its non-deformed or non-stressed configuration. This causes the volume of the haptic fluid chambers 120 to increase or return to its non-deformed volume. This increase in the volume of the haptic fluid chambers 120 causes the fluid within the optic fluid chamber 110 to be drawn out or otherwise flow out of the optic fluid chamber 110 and back into the haptic fluid chambers 120.

The optic portion 102 (any of the anterior element 106, the posterior element 108, or a combination thereof) can change shape (decrease its curvature or become flatter) in response 6 to the fluid exiting the optic fluid chamber 110 and entering the haptic fluid chambers 120. This decreases the base power or base spherical power of the AIOL 100 and allows a patient with the AIOL 100 implanted within the eye of the patient to focus on distant objects or provide for distance vision. The AIOL 100 can also be considered to be in a disaccommodated state or have undergone disaccommodation. 11

Linear chain silicone oils are oftentimes used by AIOL manufacturers to drive the shape changes needed to increase or decrease the base power of AIOLs. However, when AIOLs are filled with such linear chain silicone oils, smaller oil molecule (e.g., those with a molecular weight of less than 2000 Daltons) can enter and swell the polyacrylate bulk lens material. In some instances, the bulk lens material can swell enough to shift lens power by several diopters.

While purifying the silicone oil using a purification process, such as solvent fractionation, can address the lens swelling problem, such purification processes can increase the average molecular weight of the linear chain silicone oil as lower molecular weight components of the oil are removed through fractionation. Since the viscosity of linear chain silicone oils is directly proportional to the average molecular weight of such oils, the viscosity of such oils increases once the lower molecular weight components are removed. This inevitably decreases the rate of accommodation of an AIOL filled with such oils.

Therefore, a major technical problem faced by the applicants is how to create an ophthalmic silicone oil that does not swell the bulk polymeric lens material (thereby adversely affecting the lens power) but is still able to move and flow so that the lens can perform its accommodative functions. Moreover, the silicone oil should be produced using a method that is not overly complicated or cost-prohibitive.

The technical solution discovered by the applicants is the branched silicone oils disclosed herein. The branched polymers of the silicone oil can be substantially X-shaped or shaped as a pseudo-dendrimer. The branched silicone oil disclosed herein has several distinct advantages over linear-chain silicone oils. First, a branched silicone oil has a lower viscosity when compared to a linear-chain silicone oil at the same molecular weight. Second, branched silicone oils are physically wider than linear-chain silicone oils and, therefore, would have less of a tendency to enter the voids and holes left by the oligomers and unreacted monomers of the bulk polymeric material and swell the bulk polymeric material used to make the optic portion and haptics of the AIOL. Moreover, the branched silicone oil can be synthesized to have a refractive index that is index matched with the bulk polymeric material used to make the optic portion and haptics of the AIOL.

In some embodiments, the branched silicone oil can comprise branched polymers. At least one of the branched polymers can comprise a cyclosiloxane core and three or more linear polysiloxane arms linked or bonded to the cyclosiloxane core.

In some embodiments, the cyclosiloxane core can comprise a cyclotrisiloxane ring. In other embodiments, the cyclosiloxane core can comprise a cyclotetrasiloxane ring.

In these embodiments, the linear polysiloxane arms can comprise a linear trisiloxane backbone molecule with phenyl and methyl groups attached to the linear trisiloxane backbone molecule. In other embodiments, the linear polysiloxane arms can comprise a linear tetrasiloxane backbone molecule with phenyl and methyl groups attached to the linear tetrasiloxane backbone molecule.

In some embodiments, the branched silicone oil can have a viscosity of less than about 2,400 centipoise (cps), as measured at 25° C. In certain embodiments, the branched silicone oil can have a viscosity of between about 1,000 cps to 2,400 cps, as measured at 25° C.

The ratio of methyl groups to phenyl groups in the branched polymers can determine the refractive index of the branched silicone oil. In certain embodiments, the ratio of methyl groups to phenyl groups can be adjusted to index match the refractive index of the bulk polymeric material making up the optic portion and haptics of the AIOL. As previously discussed, the refractive index of the bulk polymeric material can be less than 1.53 or between about 1.48 and 1.53. In some embodiments, the refractive index of the branched silicone oil comprising the branched polymers disclosed herein can also be less than 1.53 or between about 1.48 and 1.53.

In some embodiments, the ratio of methyl groups to phenyl groups in the branched polymers can be about 2:1 to allow the refractive index of the branched silicone oil to match the refractive index of the bulk polymeric material making up the optic portion and haptics of the AIOL.

In some embodiments, the core ring structure of the cyclosiloxane core can comprise a plurality of ring segments with a polysiloxane arm (Rₐᵣₘ) linked to each of the ring segments. Each of the ring segments can have the structure corresponding to Formula I below:

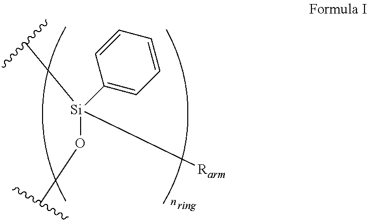

In some embodiments, n_{ring} can be an integer greater than or equal to 3.

Rₐᵣₘ can have the structure corresponding to Formula II below:

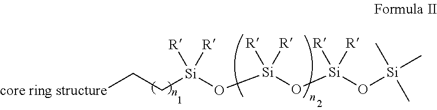

In some embodiments, n₁ can be the integer 1 or 2 and n₂ can be the integer 1 or 2. In these and other embodiments, R′ can be a phenyl group or methyl group. In some embodiments, the ratio of methyl groups to phenyl groups can be about 2:1.

In certain embodiments, R′ can be a phenyl group having one or more functional groups coupled to the phenyl group. For example, the functional groups can be selected from the group consisting of a t-butyl functional group, a sec-butyl functional group, and an isopropyl group.

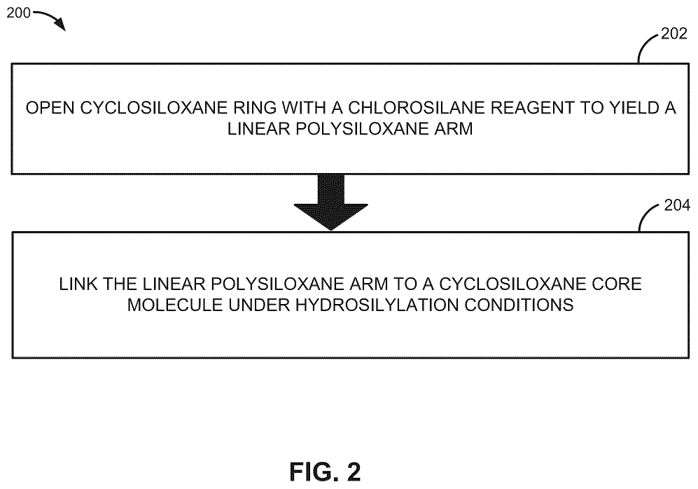

FIG. 2 illustrates a method 200 of synthesizing an ophthalmic silicone oil comprising branched polymers. For example, the method 200 can be used to synthesize the branched polymers disclosed herein.

The method 200 can comprise preparing the arms of the branched polymer by opening a cyclosiloxane ring with a chlorosilane reagent to yield a linear polysiloxane arm molecule in step 202.

In some embodiments, the cyclosiloxane ring can be a cyclotrisiloxane. In other embodiments, the cyclosiloxane ring can be selected from the group consisting of a cyclotetrasiloxane, a cyclopentasiloxane, a cyclohexasiloxane, a cycloheptasiloxane, and a cyclooctasiloxane.

The cyclosiloxane ring can comprise a plurality of phenyl groups and methyl groups coupled or attached to the cyclosiloxane ring. In some embodiments, the ratio of methyl groups to phenyl groups can be about 2:1. In one example embodiment, the cyclosiloxane ring can be a diphenyl tetramethyl cyclotrisiloxane.

In some embodiments, the chlorosilane reagent can be an allyl functional silane such as an allylchlorodimethylsilane. In other embodiments, the chlorosilane reagent can be a chlorotrimethylsilane. In these embodiments, the ring opening reaction can be performed at a reaction temperature of between about 40° C. and 50° C.

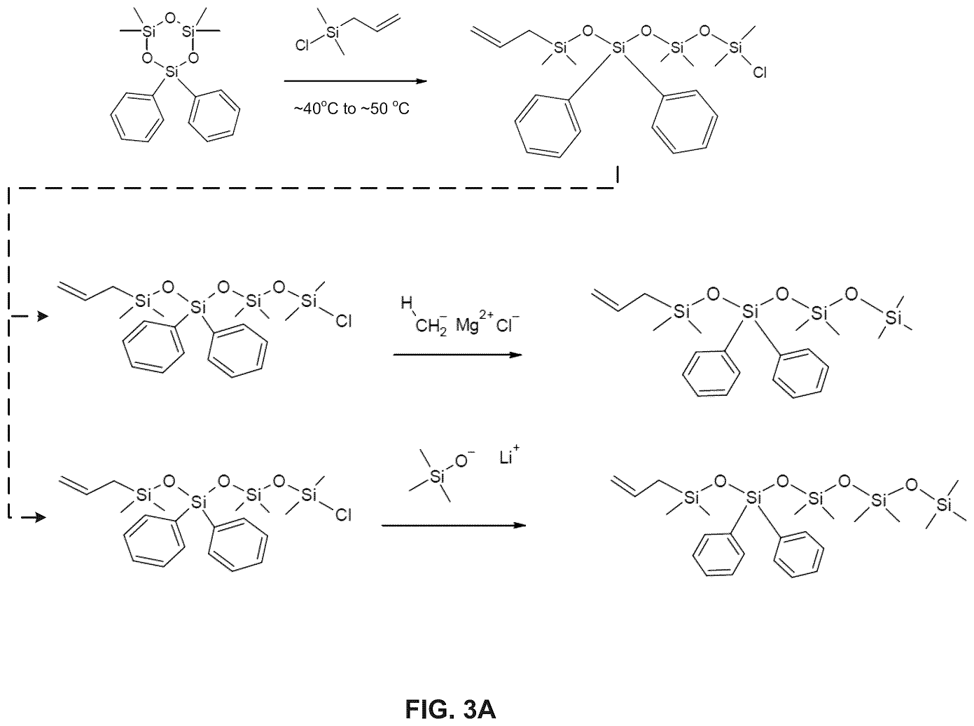

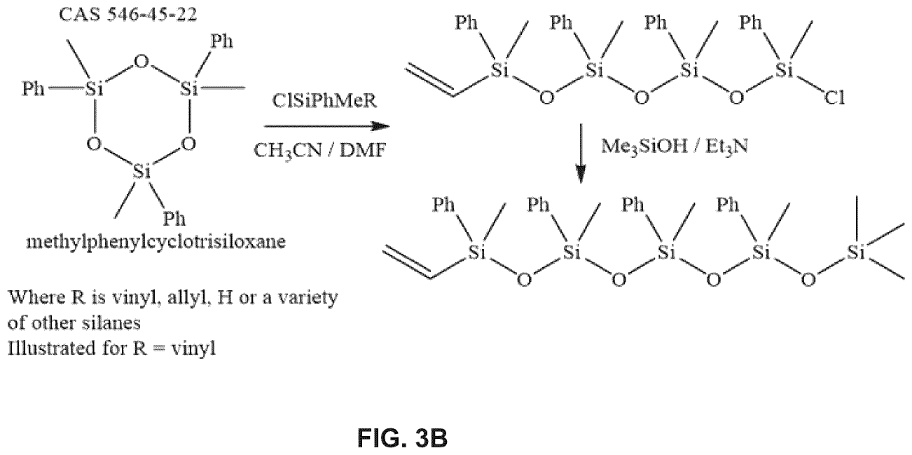

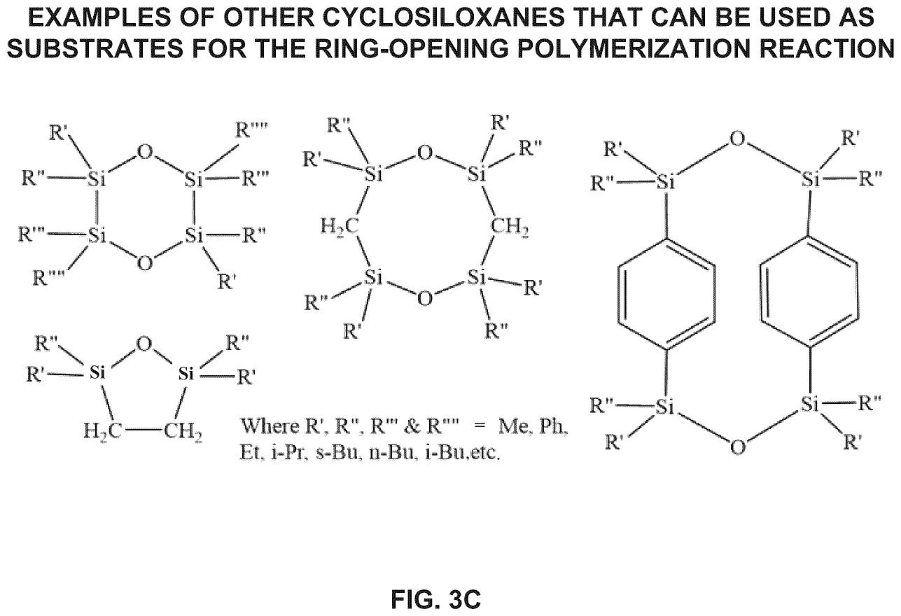

FIG. 3A illustrates examples of reaction steps for preparing arms of the branched polymer. As shown in FIG. 3A, a diphenyl tetramethyl cyclotrisiloxane ring can be opened in a ring-opening polymerization reaction with a chlorosilane reagent (for example, an allylchlorodimethylsilane (C₅H₁₁ClSi)) to yield an intermediate linear polysiloxane. The intermediate linear polysiloxane can comprise an allyl functional group on one end and a chlorine on the other end. This ring opening reaction can be performed at a reaction temperature of between about 40° C. and 50° C.

The ring opening reaction can be performed in a solvent comprising N,N-dimethyl-formamide (DMF), acetonitrile (CH₃CN), and, optionally, octamethylcyclotetrasiloxane (also known as D4).

Examples of the ring opening reaction disclosed herein can also be found in Suzuki, Takashi, Hajime Tanaka, and Toshio Nishi. “Miscibility and transesterification in bisphenol A polycarbonate/poly (ethylene terephthalate) blends.” Polymer 30.7 (1989): 1287-1297 and Cella, James A., and John C. Carpenter. “Procedures for the preparation of silanols.” Journal of Organometallic Chemistry 480.1-2 (1994): 23-26, the contents of which are incorporated herein by reference in their entireties.

Since chlorosilanes are susceptible to hydrolysis, the chlorine on the intermediate linear polysiloxane can be replaced with a methyl group by reacting the intermediate linear polysiloxane with a Grignard reagent to yield an embodiment of the linear polysiloxane arm with three siloxane units and an allyl functional group on one end. For example, as shown in FIG. 3A, the Grignard reagent can be methyl magnesium chloride (CH₃MgCl).

General procedures for the Grignard reaction can be found in, for example, B. Arkles. Grignard Reagents and Silanes. Reprinted from G. Silverman and P. Rakita. Handbook of Grignard Reagents (1996): 667-675, the content of which is incorporated herein by reference in its entirety.

Other reagents can also be used. Such reagents can be in the form of R—Mg—X where R is an alkyl or aromatic. When R is an alkyl group, the R can be a methyl, an ethyl, a propyl, an isopropyl, a butyl, an isobutyl, a sec-butyl, a tert-butyl, a pentyl, an isopentyl, a hexyl, a heptyl, an octyl, a nonyl, a decyl, a nonyl, an undecyl, a dodecyl, a tridecyl, a tetradecyl, a pentadecyl, a hexadecyl, a heptadecyl, an octadecyl, a nonadecyl, or an icosane. The alkyl groups can be branched, linear, substitute, or unsubstituted.

In some embodiments, an alkyl lithium (R—Li) or a dialkyl magnesium (R₂Mg) can also be used.

Alternatively, the chlorine containing intermediate linear polysiloxane can be functionalized with a silanolate reagent to produce an embodiment of the linear polysiloxane arm with four siloxane units and an allyl functional group on one end. For example, as shown in FIG. 3A, an embodiment of the linear polysiloxane arm with four siloxane units can be produced by reacting the intermediate linear polysiloxane with a silanolate such as a lithium trimethylsilanolate (C₃H₉LiOSi) or a potassium trimethylsilanolate (C₃H₉KOSi). The reaction can also be performed in a solvent comprising triethylamine (TEA or Et₃N).

FIG. 3B illustrates another example of reaction steps for preparing arms of the branched polymer. As shown in FIG. 3B, a triphenyltrimethylcyclotrisiloxane (CAS: 546-45-22) can be opened with a chlorosilane reagent to yield an intermediate linear polysiloxane. For example, the chlorosilane reagent can be an allylchlorodimethylsilane or a chloro (dimethyl) vinylsilane.

This ring opening reaction can be performed at a reaction temperature of between about 40° C. and 50° C. The ring opening reaction can be performed in a solvent comprising acetonitrile (CH₃CN), N,N-dimethyl-formamide (DMF), and, optionally, octamethylcyclotetrasiloxane (also known as D4).

As shown in FIG. 3B, the intermediate linear polysiloxane can be functionalized with a silanolate reagent to produce an embodiment of the linear polysiloxane arm with five siloxane units and a vinyl or allyl functional group on one end. For example, as shown in FIG. 3B, an embodiment of the linear polysiloxane arm with five siloxane units can be produced by reacting the intermediate linear polysiloxane with a silanolate such as a potassium 9 trimethylsilanolate (C₃H₉KOSi) or a lithium trimethylsilanolate (C₃H₉LiOSi). The reaction can also be performed in a solvent comprising triethylamine (TEA or Et₃N).

FIG. 3C illustrates examples of other cyclosiloxanes that can be used as monomers or substrates for the ring-opening polymerization reactions disclosed herein. For example, suitable monomers can comprise any cyclic compound containing reactive Si—O—Si groups in its ring. Any of the R′, R″, R″, or R′″ groups shown in FIG. 3C can be an alkyl group such as a methyl, an ethyl, a propyl, an isopropyl, a butyl, an isobutyl, a sec-butyl, a tert-butyl, a pentyl, an isopentyl, a hexyl, a heptyl, an octyl, a nonyl, a decyl, a nonyl, an undecyl, a dodecyl, a tridecyl, a tetradecyl, a pentadecyl, a hexadecyl, a heptadecyl, an octadecyl, a nonadecyl, or an icosane. The alkyl groups can be branched, linear, substitute, or unsubstituted.

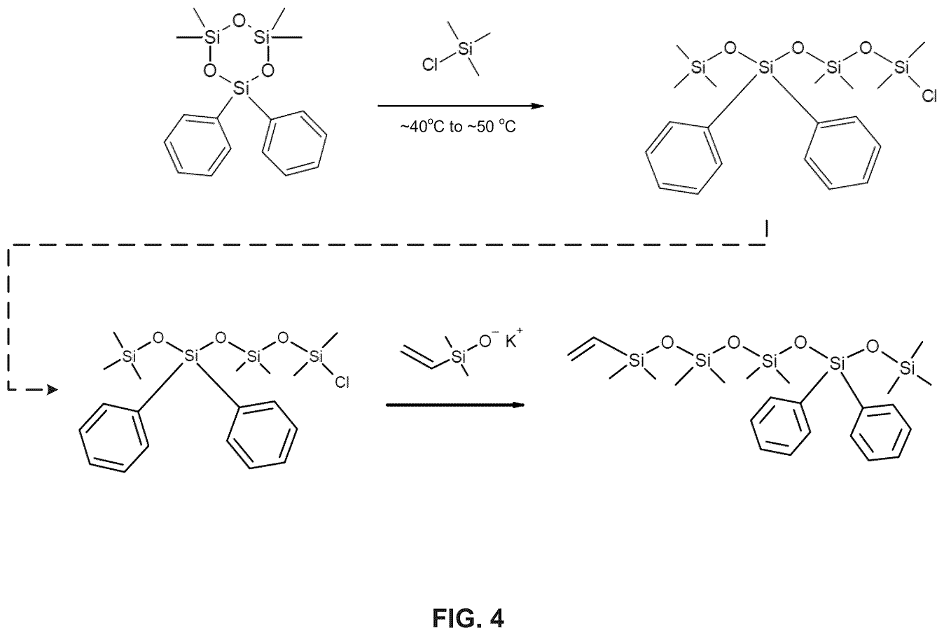

FIG. 4 illustrates another example of reaction steps for preparing arms of the branched polymer. As shown in FIG. 4, a diphenyl tetramethyl cyclotrisiloxane ring can be opened with 21 a trimethyl silane to produce an intermediate linear polysiloxane with a chlorine on one end. For example, the diphenyl tetramethyl cyclotrisiloxane ring can be opened with chlorotrimethylsilane (C₃H₉SiCl). This ring opening reaction can be performed at a reaction temperature of between about 40° C. and 50° C.

The ring opening reaction can be performed in a solvent comprising N,N-dimethyl-formamide (DMF), acetonitrile (CH₃CN), and, optionally, octamethylcyclotetrasiloxane (also known as D4).

As shown in FIG. 4, an embodiment of the linear polysiloxane arm with four siloxane units and a vinyl group (or an allyl group) can be produced by reacting the chlorine containing intermediate linear polysiloxane with a silanolate such as a potassium dimethyl (vinyl) silanolate (C₄H₉KOSi). Other silanolates can also include a potassium trimethylsilanolate (C₃H₉KOSi) or a lithium trimethylsilanolate (C₃H₉LiOSi). The reaction can also be performed in a solvent comprising triethylamine (TEA or Et₃N).

Referring back to FIG. 2, the method 200 of synthesizing the ophthalmic silicone oil can also comprise linking the linear polysiloxane arm to a cyclosiloxane core molecule in a hydrosilylation reaction or under hydrosilylation conditions in step 204. In some embodiments, the hydrosilylation reaction can involve a platinum-based catalyst such as a Karstedt catalyst.

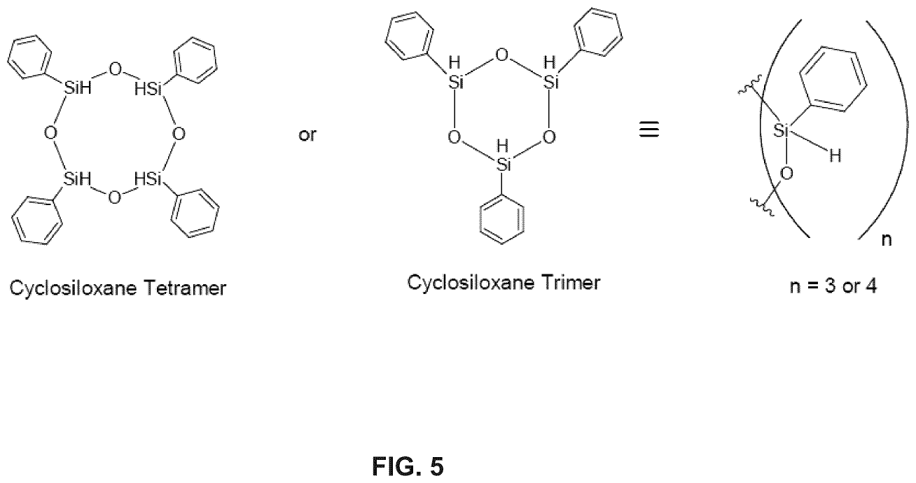

FIG. 5 illustrates different embodiments of cyclosiloxane core molecules. For example, the cyclosiloxane core molecule can be a cyclotrisiloxane molecule or a cyclosiloxane trimer. The cyclosiloxane core molecule can also be a cyclotetrasiloxane molecule or a cyclosiloxane tetramer.

Although not shown in FIG. 5, in some embodiments, the cyclosiloxane core molecule can be selected from the group consisting of a cyclopentasiloxane, a cyclohexasiloxane, a cycloheptasiloxane, and a cyclooctasiloxane.

Moreover, although FIG. 5 illustrates phenyl groups coupled to the siloxane units of the cyclosiloxane core molecule, it is contemplated by this disclosure that one or more of the phenyl groups can be replaced by methyl groups.

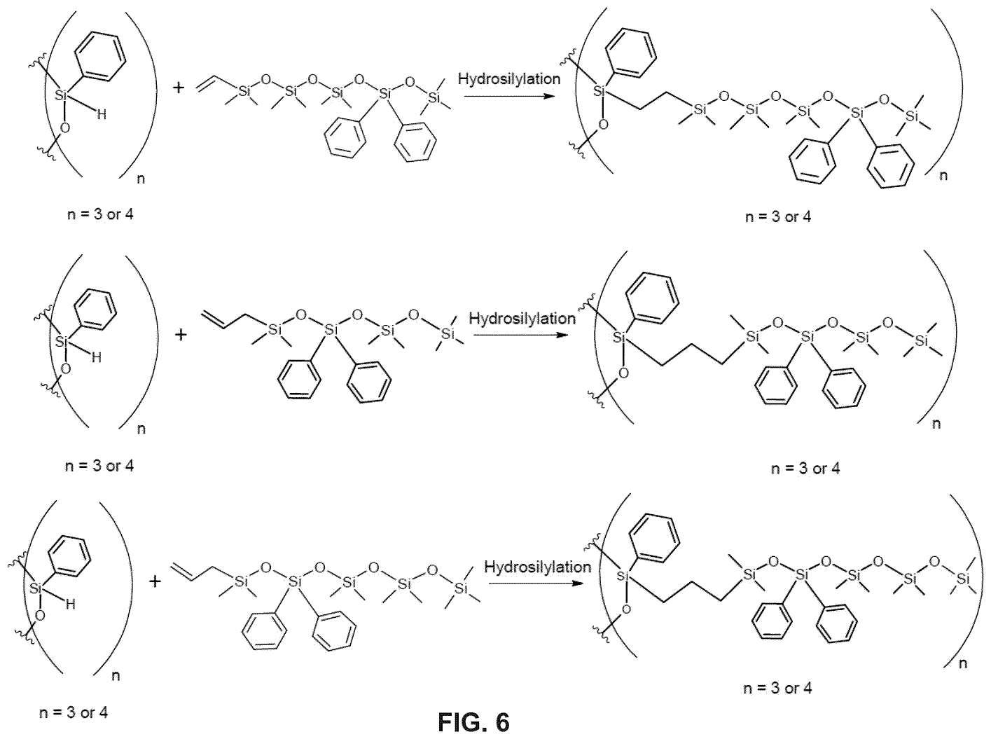

FIG. 6 illustrates that the linear polysiloxane arms can be linked to the cyclosiloxane core under hydrosilylation conditions or using a hydrosilylation reaction. In some embodiments, the hydrosilylation reaction can involve a platinum-based catalyst such as a Karstedt catalyst. More specifically, FIG. 6 illustrates that the allyl or vinyl functional groups on the linear polysiloxane arms can react with the siloxane units on the cyclosiloxane core molecule under hydrosilylation conditions to form the branched polymer comprising a plurality of branch arms. For example, the branched polymers can comprise the cyclosiloxane core and three or four linear polysiloxane arms linked to the cyclosiloxane core.

Examples of the hydrosilylation reaction disclosed herein can also be found in, for example, Naganawa, Yuki, et al. “Hydrosilylation reactions of functionalized alkenes.” Tetrahedron Letters 61.11 (2020): 151513 and Hofmann, Robin J., Matea Vlatković, and Frank Wiesbrock. “Fifty years of hydrosilylation in polymer science: A review of current trends of low-cost transition-metal and metal-free catalysts, non-thermally triggered hydrosilylation reactions, and industrial applications.” Polymers 9.10 (2017): 534, the contents of which are incorporated herein by reference in their entireties.

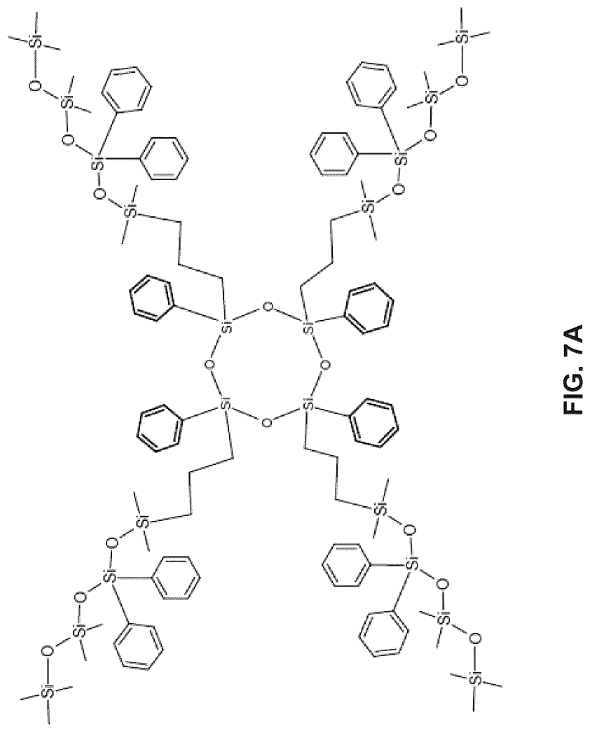

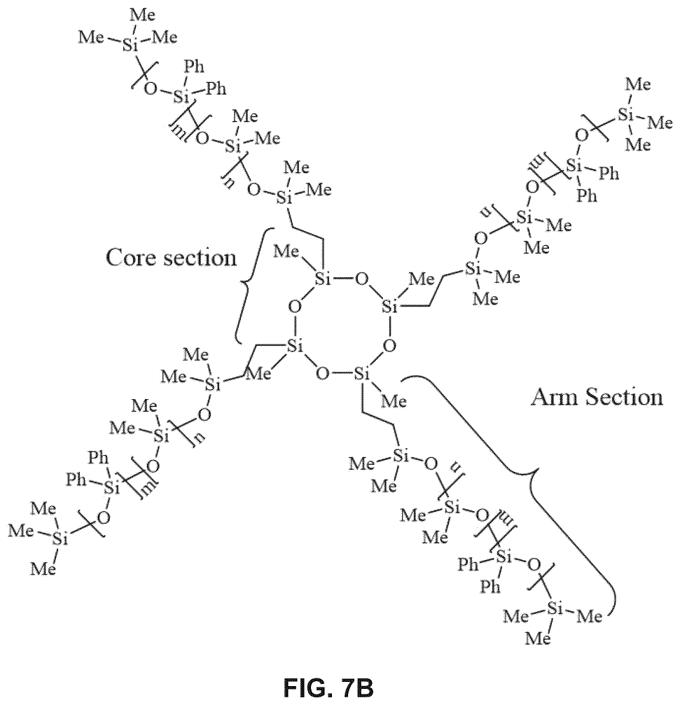

FIG. 7A illustrates one embodiment of a substantially X-shaped branched polymer synthesized using the methods disclosed herein. For example, the substantially X-shaped branched polymer can comprise a cyclosiloxane core molecule (e.g., a cyclotetrasiloxane molecule) and a plurality of linear polysiloxane arms linked to the cyclosiloxane core under hydrosilylation conditions or using a hydrosilylation reaction.

As previously discussed, the branched polymer can be used as an ophthalmic silicone oil for driving the accommodation of an AIOL (e.g., the AIOL 100 shown in FIGS. 1A-1C) or a non-accommodating fluid-adjustable IOL. Although FIG. 7A illustrates an embodiment of the branched polymer with four arms, it is contemplated by this disclosure that the branched polymer can comprise three arms, five arms, six arms, seven arms, or eight arms. Moreover, although FIG. 7A illustrates two phenyl groups attached to each arm, it is contemplated by this disclosure that one or more of the methyl groups attached to each arm can be replaced by phenyl groups.

As previously mentioned, the ratio of methyl groups or dimethylsiloxane groups to phenyl groups or diphenylsiloxane groups can be adjusted to index match the refractive index of the silicone oil with the refractive index of the bulk polymeric material making up the optic portion and haptics of the AIOL.

FIG. 7B illustrates another embodiment of a substantially X-shaped branched polymer. For example, the substantially X-shaped branched polymer can comprise a cyclosiloxane core molecule and a plurality of linear polysiloxane arms linked to the cyclosiloxane core under hydrosilylation conditions or using a hydrosilylation reaction.

As shown in FIG. 7B, the ratio of methyl groups or dimethylsiloxane groups (n) to phenyl groups or diphenylsiloxane groups (m) on the arms can be adjusted to index match the refractive index of the silicone oil with the refractive index of the bulk polymeric material making up the optic portion and haptics of the AIOL.

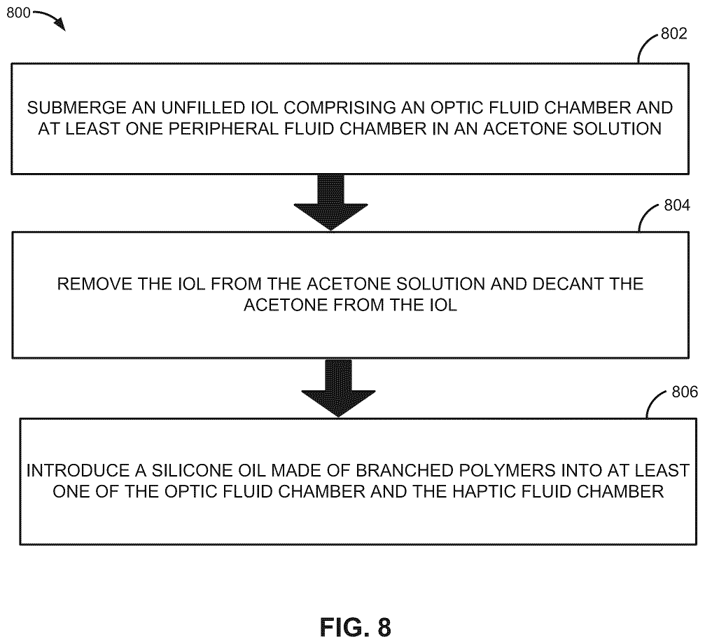

FIG. 8 illustrates a method 800 of manufacturing a fluid-filled IOL. For example, the fluid-filled intraocular lens can be an AIOL or a non-accommodating fluid-adjustable IOL.

The optic portion and peripheral portion (e.g., haptic(s)) of a fluid-filled IOL can often be made using a bulk polymerization process such as a bulk acrylate polymerization process. In general, bulk polymerization converts small monomers into polymers without solvent. While bulk polymerization produces mostly high molecular weight polymers, it also produces a small amount of lower molecular weight oligomers and unreacted monomers. These oligomers are not bonded to the bulk polymer and are simply trapped inside the bulk polymer. One technical problem faced by the applicants is that if these non-bonded oligomers and unreacted monomers were allowed to remain in the IOL, they would gradually migrate out and adversely affect the lens quality. One technical solution discovered by the applicant for removing the non-bonded oligomers and unreacted monomers is the method 800 disclosed herein.

The method 800 can comprise submerging an unfilled IOL (e.g., an AIOL or a non-accommodating fluid-adjustable IOL) made, in part, of a polyacrylate material or made from a bulk polymerization process (e.g., a bulk acrylate polymerization process) in acetone (100% v/v) in step 802. The IOL can comprise an optic fluid chamber and at least one peripheral fluid chamber (e.g., a haptic fluid chamber).

For example, one unfilled IOL can be submerged in a container or a well of a multi-well plate comprising at least 20 mL (or between 20 mL and 30 mL) of acetone. Also, for example, multiple unfilled IOLs can be submerged in a container comprising 100 mL (or greater than 100 mL) of acetone.

When an IOL made of polyacrylate is submerged in the acetone, the acetone molecules diffuse throughout and occupy spaces between polymers strands. In acetone, the IOL appears to inflate but the lens structure is bloated or swelled with acetone. While the lens is in its swelled state, the acetone molecules diffuse in and out of the polymer in dynamic equilibrium with the acetone surrounding the polyacrylate. Moreover, when the lens is in its swelled state, the acetone molecules carry the oligomers and unreacted monomers out of the polyacrylate matrix. Once outside, these lower weight molecules do not re-enter the polyacrylate.

The method 800 can further comprise removing the IOL from the acetone and decanting the acetone from the IOL in step 804.

The unfilled IOL can be submerged in the acetone for at least 19 hours in total. For example, the IOL can be submerged in the acetone for between about 19 hours to about 24 hours in total. Old acetone can be decanted or poured out and new acetone can be introduced (multiple times) during this 19 hour to 24 hour period.

At the end of this period, once all of the acetone is decanted or otherwise removed a final time, the unfilled IOL can be dried under vacuum.

The method 800 can further comprise introducing a silicone oil made of branched polymers into at least one of the optic fluid chamber and the peripheral fluid chamber in step 806. The silicone oil introduced can be the silicone oil disclosed herein. For example, the silicone oil can comprise branched polymers comprising a cyclosiloxane core and three or more linear polysiloxane arms linked or bonded to the cyclosiloxane core. For example, the silicone oil can be introduced into the IOL by being injected into at least one of the optic fluid chamber and the peripheral fluid chamber. In some embodiments, the silicone oil can be injected into the optic fluid chamber and/or the peripheral fluid chamber of the IOL using a syringe.

In certain embodiments, the silicone oil can be introduced into the IOL by being injected into at least one of the optic fluid chamber and the peripheral fluid chamber under vacuum.

In some embodiments, the cyclosiloxane core can comprise a cyclotrisiloxane ring. In other embodiments, the cyclosiloxane core can comprise a cyclotetrasiloxane ring.

In these embodiments, the linear polysiloxane arms can comprise a linear trisiloxane backbone molecule with phenyl and methyl groups attached to the linear trisiloxane backbone molecule. In other embodiments, the linear polysiloxane arms can comprise a linear tetrasiloxane backbone molecule with phenyl and methyl groups attached to the linear tetrasiloxane backbone molecule. In certain embodiments, the ratio of methyl groups to phenyl groups in the branched polymers can be about 2:1.

In some embodiments, the silicone oil can have a viscosity of less than about 2,400 cps, as measured at 25° C. In certain embodiments, the silicone oil can have a viscosity of between about 1,000 cps to 2,400 cps, as measured at 25° C. The refractive index of a bulk polymeric material making up an optic portion of the

IOL can be less than 1.53 or between about 1.48 and 1.53. In some embodiments, the refractive index of the silicone oil comprising the branched polymers disclosed herein can also be less than 1.53 or between about 1.48 and 1.53.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween. All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention. 6

Reference to a singular item, includes the possibility that there are plural of the same items present. More specifically, as used herein and in the appended claims, the singular forms 8 “a,” “an,” “said” and “the” include plural referents unless the context clearly dictates 9 otherwise. It is further noted that the claims may be drafted to exclude any optional element.

As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps.

The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 g” can be interpreted to mean “1.0 g” or between “0.9 g and 1.1 g.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An ophthalmic silicone oil comprising branched polymers, wherein at least one of the branched polymers comprises:
a cyclosiloxane core, and
three or more linear polysiloxane arms linked to the cyclosiloxane core.

2. The ophthalmic silicone oil of claim 1, wherein the cyclosiloxane core comprises a cyclotrisiloxane ring.

3. The ophthalmic silicone oil of claim 1, wherein the cyclosiloxane core comprises a cyclotetrasiloxane ring.

4. The ophthalmic silicone oil of claim 1, wherein at least one of the linear polysiloxane arms comprises a linear trisiloxane backbone.

5. The ophthalmic silicone oil of claim 1, wherein at least one of the linear polysiloxane arms comprises a linear tetrasiloxane backbone.

6. The ophthalmic silicone oil of claim 1, wherein the branched polymers are substantially X-shaped.

7. The ophthalmic silicone oil of claim 1, wherein the ophthalmic silicone oil has a viscosity between about 1000 to 2400 centipoise (cps), as measured at 25° C.

8. The ophthalmic silicone oil of claim 1, wherein the ophthalmic silicone oil has a refractive index of less than 1.53.

9. A branched polymer, comprising:
a core ring structure comprising a plurality of ring segments and a polysiloxane arm (Rₐᵣₘ) linked to each of the ring segments,
wherein each of the ring segments has a structure corresponding to Formula I below: 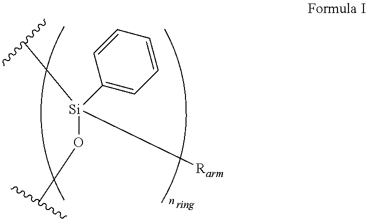
wherein n_{ring} is an integer ≥3; and
wherein Rₐᵣₘ has a structure corresponding to Formula II below: 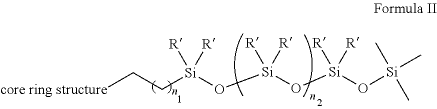
wherein n₁ is an integer=1 or 2, n₂ is an integer=1 or 2, wherein R′ is a phenyl group or a methyl group.

10. The branched polymer of claim 9, wherein the ratio of methyl groups to phenyl groups is 2:1.

11. The branched polymer of claim 9, wherein the branched polymers are substantially X-shaped.

12. A method for synthesizing an ophthalmic silicone oil comprising branched polymers, comprising:
opening a cyclosiloxane ring with a chlorosilane reagent to yield a linear polysiloxane arm; and
linking the linear polysiloxane arm to a cyclosiloxane core molecule in a hydrosilylation reaction.

13. The method of claim 12, wherein the cyclosiloxane ring is a diphenyl tetramethyl cyclotrisiloxane.

14. The method of claim 12, wherein the chlorosilane reagent is an allylchlorodimethylsilane, and wherein the ring opening reaction is performed at a reaction temperature of between about 40° C. and 50° C.

15. The method of claim 12, wherein the chlorosilane reagent is a chlorotrimethylsilane, and wherein the ring opening reaction is performed at a reaction temperature of between about 40° C. and 50° C.

16. The method of claim 12, wherein opening the cyclosiloxane ring yields an intermediate linear polysiloxane, wherein the method further comprises reacting the intermediate linear polysiloxane with a Grignard reagent to yield the linear polysiloxane arm.

17. The method of claim 12, wherein opening the cyclosiloxane ring yields an intermediate linear polysiloxane, wherein the method further comprises reacting the intermediate linear polysiloxane with a silanolate reagent to yield the linear polysiloxane arm.

18. The method of claim 17, wherein the silanolate reagent is a lithium trimethylsilanolate.

19. The method of claim 17, wherein the silanolate reagent is a potassium dimethyl (vinyl) silanolate.

20. (canceled)

21. A method of manufacturing a fluid-filled intraocular lens, comprising:
submerging an unfilled intraocular lens made in part of a polyacrylate material in acetone, wherein the intraocular lens comprises an optic fluid chamber and at least one peripheral fluid chamber;
removing the intraocular lens from the acetone and decanting the acetone from the intraocular lens; and
introducing a silicone oil made of branched polymers into at least one of the optic fluid chamber and the at least one peripheral fluid chamber.

22.-30. (canceled)
